(19) United States
(12) Patent Application Publication
(10) Pub. No.: US 2026/0061095 A1
(43) Pub. Date: Mar. 5, 2026

(54) BONE COLLAGEN SCAFFOLD MATERIAL LOADED WITH MULTIPLE GROWTH FACTORS AND A PREPARATION METHOD THEREOF

(71) Applicants: Beijing Yiling Biotechnology Co., Ltd., Beijing (CN); Xiling (Zhenjiang) Medical Science Co., Ltd., Beijing (CN)

(72) Inventors: Hui Sun, Beijing (CN); Lixin Xu, Beijing (CN); Manqi Yin, Beijing (CN); Wenhao Zhang, Beijing (CN); Feixiang Cai, Beijing (CN)

(21) Appl. No.: 19/283,269

(22) Filed: Jul. 29, 2025

(30) Foreign Application Priority Data
Aug. 28, 2024 (CN) 202411192704.3

(51) Int. Cl. A61L 27/24; A61L 27/36; A61L 27/54

(52) U.S. Cl. CPC A61L 27/24; A61L 27/3608; A61L 27/54; A61L 2430/02

(57) ABSTRACT

A bone collagen scaffold material loaded with multiple growth factors, which includes an acellular matrix for promoting growth of oral osteoblasts and multiple growth factors loaded on the acellular matrix; the acellular matrix is a bone collagen scaffold obtained from natural animal bone containing collagen; the multiple growth factors are FGF, VEGF, TGF-β, and BMP. The bone collagen scaffold material of the invention has a higher collagen content through secondary freeze-drying, making surface of the bone collagen scaffold material have a suitable pore size, guaranteeing mechanical properties and improve bone induction ability, being suitable for loading multiple growth factors and prolonging the degradation time to adapt to the therapeutic effect of periodontitis.

### 1. TECHNICAL FIELD

The invention belongs to the technical field of biomedicine, specifically relates to a bone collagen scaffold material loaded with multiple growth factors and a preparation method thereof.

### 2. BACKGROUND ART

Periodontitis, also known as destructive periodontal disease, is a chronic inflammatory disease mainly caused by the destruction of periodontal tissues by bacteria in dental plaque. Periodontitis can lead to the destruction of periodontal supporting tissues (gingiva, peridental membrane, alveolar bone, and cementum), the formation of periodontal pockets (small pockets formed by widening of the gap between teeth and gingiva), attachment loss, and alveolar bone resorption. As the disease progresses, teeth gradually loosen, and gingival recession can eventually lead to tooth loss. Oral implant restoration is currently the preferred restoration method for patients with missing teeth, but the alveolar bone in the implant area often undergoes atrophy, resulting in insufficient bone mass, which can significantly affect the restoration effect.

According to incomplete statistics, the proportion of adults with periodontitis in China is as high as over 60%. Currently, guided bone tissue regeneration technology is the mainstream adjuvant treatment procedure, mainly by implanting bone substitutes or other biological materials at the site of periodontal bone loss to promote new bone formation. Bone substitute materials currently used in clinical practice are roughly divided into synthetic materials and biological bone materials. Among them, synthetic materials have the problem that the degradation rate is difficult to control, leading to poor bone regeneration effects. Most clinically applied materials are biological bone materials, which are pure inorganic three-dimensional xenogeneic carbonate apatite crystals extracted after removing organic components from bovine bone, such as the finished product Bio-oss artificial bone powder provided by Osseus Technologies & Systems, LLC. (US) of Geistlich (Switzerland) and This bone powder has a single component without any organic components, suffering from poor mechanical properties and fast degradation rate.

Therefore, seeking a biological material with good mechanical properties, a degradation rate suitable for bone defect filling, and the ability to promote the growth of oral osteoblasts remains an urgent technical problem to be solved.

### 3. SUMMARY OF THE INVENTION

Aiming at the above-mentioned problems and objectives, the invention provides a bone collagen scaffold material loaded with multiple growth factors and a preparation method thereof. The bone collagen scaffold is obtained by processing natural animal bone and loaded with multiple growth factors to improve mechanical properties and bone induction ability, prolong degradation time, and adapt to the treatment cycle of periodontitis. The specific technical solutions are as follows:

First, the invention provides a bone collagen scaffold material loaded with multiple growth factors, which includes an acellular matrix for promoting growth of oral osteoblasts and multiple growth factors loaded on the acellular matrix; the acellular matrix is a bone collagen scaffold obtained from natural animal bone containing collagen; the multiple growth factors are selected from two or more of FGF, VEGF, TGF-β, and BMP.

In the aforementioned bone collagen scaffold material loaded with multiple growth factors, the bone collagen scaffold contains 80 wt %˜98 wt % of collagen, 2 wt %˜10 wt % of miscellaneous proteins, and the rest is hydroxyapatite.

Preferably, in the aforementioned bone collagen scaffold material loaded with multiple growth factors, the total protein content of the bone collagen scaffold is 84.22 wt %˜98.53 wt %.

Further preferably, in the aforementioned bone collagen scaffold material loaded with multiple growth factors, the bone collagen scaffold contains 96.74 wt % of collagen and 1.79 wt % of miscellaneous proteins, and the pore size on its surface is 60˜70 μm.

In the aforementioned bone collagen scaffold material loaded with multiple growth factors, the material contains four growth factors, namely FGF, VEGF, TGF-β, and BMP, with the loading amounts as follows:
FGF: 20˜27 ng/g,
VEGF: 18˜25 ng/g,
TGF-β: 26˜32 ng/g,
BMP: 24˜30 ng/g.

In the aforementioned bone collagen scaffold material loaded with multiple growth factors, the wet compressive strength of the material is 0.0120˜0.0221 MPa, and the dry compressive strength is 1.25˜2.90 MPa.

Secondly, the invention provides a preparation method for the aforementioned bone collagen scaffold material loaded with multiple growth factors, including the following steps:
1) preparing a bone collagen scaffold: removing surface fat from animal bone, pouring it into a cleaning tank, and placing it in a constant-temperature oscillator for defatting: then, drying and pulverizing the bone, immersing it in a protease solution for 30˜60 min, washing it 8˜10 times with an acidic solution, filtering it out, finally soaking it in purified water for 24˜48 h, draining, and freeze-drying to obtain the bone collagen scaffold;
2) loading growth factors: secondarily pulverizing the bone collagen scaffold prepared in step 1) to a particle size of 1˜10 μm, then immersing it in a growth factor solution for 12˜24 h, centrifuging, discarding a supernatant, pouring it into a mold for freeze-drying, packaging, and sterilizing by irradiation to obtain bone collagen scaffold material.

As a preferred technical solution, in the preparation method of the aforementioned bone collagen scaffold material loaded with multiple growth factors, in step 1), when preparing the bone collagen scaffold, defatting solution is a mixture of dichloromethane or chloroform and methanol; the defatting parameters are: temperature: 5˜45° C.; rotation speed: 300˜600 rpm; the defatting solution is a mixture of dichloromethane or chloroform and methanol, with a material-to-liquid ratio of 1:3˜5.

As a preferred technical solution, in the preparation method of the aforementioned bone collagen scaffold material loaded with multiple growth factors, in step 1), when preparing the bone collagen scaffold, the acidic solution is hydrochloric acid and/or phosphoric acid with a concentration of 0.5˜1 mol/L; the purified water soaking temperature is 0° C., and the purified water is changed every 12 h during soaking: pH is detected when changing the water, and it is qualified when the pH value is 6˜7.5.

As a preferred technical solution, in the preparation method of the aforementioned bone collagen scaffold material loaded with multiple growth factors, in step 2), for loading growth factors, the concentration of the growth factor solution is:
FGF: 30˜50 ng/ml,
VEGF: 25˜40 ng/ml,
TGF-β: 40˜65 ng/ml,
BMP: 40˜65 ng/ml.

The bone collagen scaffold is added to the growth factor solution with a material-to-liquid ratio of 1:5˜10.

The soaking temperature is 0° C.

The irradiation sterilization dose is 10˜30 kGy.

The beneficial effects of the invention are:
1) the bone collagen scaffold material of the invention uses a bone collagen scaffold defatted by washing in a constant-temperature oscillator, reducing the use of chemical reagents and removing reagent residues through multiple washings, thus having better biological safety;
2) the bone collagen scaffold material of the invention improves bone induction ability and prolongs degradation time by loading growth factors. Among them, FGF promotes the synthesis of collagen and non-collagen by bone cells; VEGF effectively promotes the differentiation of osteoblasts, thus directly promoting osteogenesis; TGF-β changes the phenotype of normal fibroblasts; BMP is effective for vertical bone defects and has the strongest bone induction ability. The combined use of the four factors: VEGF and FGF promote angiogenesis; BMP-2 and TGF-β participate in bone regeneration and repair together by regulating the proliferation and differentiation of osteoblasts; VEGF improves blood supply through promoting angiogenesis, enhancing the bone induction effect of BMP-2; TGF-β can up-regulate the expression of VEGF and enhance its activity, and can jointly regulate the process of bone repair and regeneration with VEGF, FGF, and BMP-2 to achieve the purpose of promoting bone regeneration. Moreover, with the participation of new blood vessels, a better regional osteogenic effect can be achieved.
3) the bone collagen scaffold material of the invention has a higher collagen content through secondary freeze-drying, which can better promote bone regeneration. It also makes surface of the bone collagen scaffold material have a suitable pore size, making it more suitable for cell climbing, achieving the purpose of having good mechanical properties and degradation rate, being suitable for bone defect filling, and promoting the growth of oral osteoblasts.
4) the bone collagen scaffold material of the invention is sterilized by low-dose irradiation during packaging, which achieves the sterilization effect without damaging the structure of growth factors, ensuring that the mechanical properties, bone induction ability, and degradation time of the product are not affected.

### 4. BRIEF DESCRIPTION OF ACCOMPANY DRAWINGS

FIG. 1 is an SEM analysis result (500 μm) of the bone collagen scaffold material loaded with multiple growth factors of the invention.

### 5. SPECIFIC EMBODIMENT OF THE INVENTION

To make the objectives, technical solutions, and advantages of the invention clearer, the technical solutions of the invention will be clearly and completely described below in conjunction with the embodiments and the drawings.

### Embodiment 1

This Embodiment is to prepare a bone collagen scaffold material loaded with multiple growth factors. Specifically as follows:

Initially remove surface fat of animal bone, and wash the fat-removed raw material with purified water until no oil overflows. Pour the washed raw material into a cleaning tank and place it in a constant-temperature oscillator for defatting. The temperature is 30° C., the rotation speed is 500 rpm, the solution needs to completely cover the raw material (the solution is a chloroform-methanol solution, and the volume ratio of chloroform to methanol is chloroform:methanol=1:5), the material-to-liquid ratio is 1:3, soak for 22.5 h. After soaking, take it out and wash it 5 times with flowing purified water, then put it into a vacuum drying oven for 6 h. Put the defatted raw material into a pulverizer, preliminarily screen it with a filter screen, then immerse it in a protease solution for 60 min, put it into an acidic solution to wash 10 times, and filter it out. The acidic solution is hydrochloric acid with a concentration of 0.9 mol/L. Then,

soak the raw material in purified water at 0° C. for 42 h, change the purified water every 12 h, detect the pH when changing the water for the last time, the pH is 7, drain the raw material, and put it into a freeze dryer.

After freeze-drying, the raw material is secondarily pulverized to a particle size of 5 μm. Immerse the pulverized raw material in a growth factor solution. The concentration of the growth factor solution is: FGF: 32 ng/ml, VEGF: 230 ng/ml, TGF-β: 49 ng/ml, BMP: 44 ng/ml. Mix the solution evenly, the material-to-liquid ratio is 1:6, soak at 0° C. for 20 h, centrifuge, discard a supernatant, pour into a mold for freeze-drying, package, and sterilize by irradiation (15 kGy) to obtain a bone collagen scaffold material loaded with multiple growth factors.

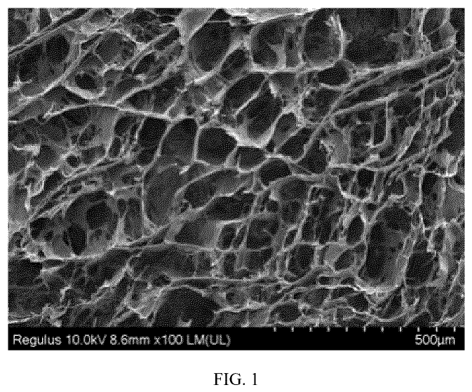

The loading amounts of growth factors in the bone collagen scaffold material are: FGF 25 ng/g, VEGF 20 ng/g, TGF-β 30 ng/g, BMP 28 ng/g. SEM analysis shows that the pore size on surface of the bone collagen scaffold material is 68 μm, as shown in FIG. 1.

### Embodiment 2

This Embodiment investigates the influence of secondary freeze-drying on the formation of the pore size on the surface of the bone collagen scaffold material and its mechanical properties.

Meanwhile, a control sample 1 prepared without secondary freeze-drying and an existing bone powder (Geistlich, specification model: 50 mg) as a control sample 2 are used. The specific preparation method is as follows:
other steps are the same as those in Embodiment 1. Without secondary pulverization after freeze-drying, detect pH after changing the water for the last time, when the pH is 7, drain the raw material, directly immerse it in the growth factor solution, directly freeze-dry it after soaking, package it, and sterilize it by irradiation.

In this Embodiment, the dry and wet compressive strengths of the prepared bone collagen scaffold material are used as evaluation indicators. The specific detection method is: pour the sample prepared in Embodiment 1, the control sample 1 prepared in this Embodiment, and the existing bone powder into the same mold (or cut) into samples of the same size 8×8×8 (unit: mm), and measure the length, width, and thickness of the sample with a vernier caliper, accurate to 0.01 mm. Use a single-column tabletop testing machine, cut upper and lower bottom surfaces of the sample in contact with the equipment flat, the area of the lower bottom surface in contact with the equipment is S (S=LW). Set the sample deformation to 5 mm in the testing machine software, and carry out test at a speed of 10 mm/min. When the testing machine stops, record the sample force value F, and calculate the compressive strength P=F/S. The results are shown in Table 1:

TABLE 1
Influence of bone collagen scaffold materials with different
surface pore sizes on their mechanical properties

| | | Pore | | Wet | | Dry | |
|---|---|---|---|---|---|---|---|
| | | Size | | Compressive | | Compressive | |
| Sample | Drying Method | (μm) | | Strength (MPa) | | Strength (MPa) | |
| Embodiment1 | Secondary Freeze-drying | 68 | μm | 0.0221 | MPa | 2.905 | MPa |
| Control Sample 1 | Primary Freeze-drying | 79 | μm | 0.0181 | MPa | 1.905 | MPa |
| Existing Bone Powder | Other Methods | 43 | μm | 0.00314 | MPa | 3.180 | MPa |

From the results in Table 1, it can be seen that the scaffold after pulverization and secondary freeze-drying has a pore size of 68 μm, with a wet compressive strength of 0.0221 MPa and a dry compressive strength of 2.90 MPa, having good porosity and suitable compactness, which can well load growth factors and provide appropriate support. The control sample 1 prepared without secondary pulverization after freeze-drying has a surface pore size of 79 μm, which is too loose, with a wet compressive strength of 0.0181 MPa and a dry compressive strength of 1.905 MPa, insufficient in support, and failing to meet the degradation time requirement. The control sample 2 prepared from existing bone powder has a surface pore size of 79 μm, which is too dense, with a wet compressive strength of 0.00314 MPa and a dry compressive strength of 3.180 MPa. Although the support is high, the degradation time is long, which is not conducive to new bone growth and cannot well match the treatment cycle of periodontitis.

### Embodiment 3

This Embodiment investigates the influence of the content of growth factors in the bone collagen scaffold material on its degradation time.

The bone collagen scaffold materials investigated in this Embodiment are the same as those in Embodiment 1 except for the different contents of growth factors, and the contents of growth factors in each bone collagen scaffold material are shown in Table 2.

A method for investigating the degradation time of the bone collagen scaffold material refers to:
YY/T0474-2004 In vitro Degradation Test for Poly-L-lactide Resins and Products for Surgical Implants,
GB/T16886.9-2001 Biological Evaluation of Medical Devices—Part 9: Framework for Qualification and Quantification of Potential Degradation Products,
GB/T16886.13-2001 Biological Evaluation of Medical Devices—Part 13: Qualification and Quantification of Degradation Products of Polymeric Medical Devices.

The specific in vitro degradation experiment process is:

The soaking solution (phosphate buffer: Sorensen buffer) is prepared with sterile secondary distilled water containing potassium dihydrogen phosphate and disodium hydrogen phosphate. The salts used to prepare the above buffer are of analytical grade and dried to constant weight.
a) 1/15 mol/L potassium dihydrogen phosphate: dissolve 9.078 g of potassium dihydrogen phosphate in each liter of water;
b) 1/15 mol/L disodium hydrogen phosphate: dissolve 11.876 g of disodium hydrogen phosphate dihydrate in each liter of water;
the soaking solution is prepared by mixing 18.2% solution a) and 81.8% solution b).

The concentration of type I collagenase added to the soaking solution is 1.25 U/ml, and the pH value of the buffer is 7.4±0.2.

Place test samples in a container, cover the samples with the prepared soaking solution, and seal the container. The test samples are completely immersed in the soaking solution, approximately 20 ml of the soaking solution is used for each sample, and a constant temperature water bath or oven is used to maintain the test samples at a physiological temperature of (37±1° C.) to simulate in vivo degradation. Record the complete degradation time of the samples, and the results are shown in Table 2.

The growth factor contents and in vivo degradation times of each bone collagen scaffold material are shown in Table 2.

TABLE 2
Influence of different growth factor contents on the
degradation time of bone collagen scaffold materials

| | Growth factor contents (ng/g) | | | | Degradation |
|---|---|---|---|---|---|
| Sample | FGF | VEGF | TGF-β | BMP | time(Day) |
| 1 | 25 | 20 | 30 | 28 | 182 |
| 2 | 25 | 18 | 26 | 28 | 178 |
| 3 | 25 | 20 | 30 | 28 | 180 |
| 4 | 25 | 20 | 30 | 24 | 175 |
| 5 | 20 | 20 | 30 | 28 | 173 |
| 6 | 20 | 18 | 26 | 24 | 168 |
| 7 | 27 | 25 | 32 | 30 | 191 |
| 8 | 15 | 15 | 20 | 20 | 93 |
| 9 | 30 | 30 | 35 | 35 | 267 |

It can be seen from the results in Table 2 that the scaffolds with growth factor contents within the range all have good degradation effects. When the content is lower than the range, the degradation time of the scaffold is shortened, making it difficult to meet clinical needs. When the content is higher than the range, the degradation time of the scaffold is too long, and there is a possibility of cell proliferation, which is not conducive to the subsequent treatment of patients.

### Embodiment 4

The Embodiment detects the biological safety of the bone collagen scaffold material prepared in Embodiment 1.

In this Embodiment, the prepared bone collagen scaffold material is used as a test sample, and polar and non-polar extraction solutions are used for extraction. The presence of toxic substances in the solution at different extraction times is checked as an index. The detection method is carried out according to GB/T16886.11-2021Biological Evaluation of Medical Devices—Part 11: Systemic Toxicity Tests, and the results are shown in Table 3.

TABLE 3
Influence of the biological safety of the bone collagen scaffold
material prepared by the preparation method of the invention

| Observation | | | Polar Extraction | Non-polar Extraction |
|---|---|---|---|---|
| Time | | Sample | Solution | Solution |
| Immediately | | Embodiment1 | AN | AN |
| 4 | h | Embodiment1 | AN | AN |
| 24 | h | Embodiment1 | AN | AN |
| 48 | h | Embodiment1 | AN | AN |
| 72 | h | Embodiment1 | AN | AN |

Note:
AN = normal appearance

It can be seen from the results in Table 3 that the bone collagen scaffold material prepared by the preparation method of the invention has good biological safety and can be used for the treatment of periodontitis guided bone tissue regeneration.

It is apparent to those skilled in the art that the invention is not limited to the details of the embodiments described above, and that the invention may be embodied in other specific forms without departing from its spirit or essential characteristics. Accordingly, the embodiments should be regarded as illustrative rather than restrictive in all respects. Furthermore, it should be understood that although this specification is described in terms of embodiments, it is not limited to only one technical solution. Such description in the specification is solely for clarity, and those skilled in the art should consider the specification as a whole. The technical solutions in the embodiments may also be appropriately combined to form other implementations that would be understood by those skilled in the art.

1. A bone collagen scaffold material loaded with multiple growth factors, wherein the bone collagen scaffold material includes an acellular matrix for promoting growth of oral osteoblasts and multiple growth factors loaded on the acellular matrix;
the acellular matrix is a bone collagen scaffold obtained from natural animal bone containing collagen;
the multiple growth factors are selected from two or more of FGF, VEGF, TGF-β, and BMP.

2. The bone collagen scaffold material loaded with multiple growth factors according to claim 1, wherein the bone collagen scaffold contains 80 wt %˜98 wt % of collagen, 2 wt %˜10 wt % of miscellaneous proteins, and the rest is hydroxyapatite.

3. The bone collagen scaffold material loaded with multiple growth factors according to claim 2, wherein the total protein content of the bone collagen scaffold is 84.22 wt %˜98.53 wt %.

4. The bone collagen scaffold material loaded with multiple growth factors according to claim 3, wherein the bone collagen scaffold contains 96.74 wt % of collagen and 1.79 wt % of miscellaneous proteins, and the pore size on its surface is 60˜70 μm.

5. The bone collagen scaffold material loaded with multiple growth factors according to claim 1, wherein the bone collagen scaffold material contains four growth factors, namely FGF, VEGF, TGF-β, and BMP, with the loading amounts as follows:
FGF: 20˜27 ng/g,
VEGF: 18˜25 ng/g,
TGF-β: 26˜32 ng/g,
BMP: 24˜30 ng/g.

6. The bone collagen scaffold material loaded with multiple growth factors according to claim 1, wherein the wet compressive strength of the material is 0.0120˜0.0221 MPa, and the dry compressive strength is 1.25˜2.90 MPa.

7. A preparation method for the bone collagen scaffold material loaded with multiple growth factors according to claim 1, including the following steps:
1) preparing a bone collagen scaffold: removing surface fat from animal bone, pouring it into a cleaning tank, and placing it in a constant-temperature oscillator for defatting; then, drying and pulverizing the bone, immersing it in a protease solution for 30˜60 min, washing it 8˜10 times with an acidic solution, filtering it out, finally soaking it in purified water for 24˜48 h, draining, and freeze-drying to obtain the bone collagen scaffold;
2) loading growth factors: secondarily pulverizing the bone collagen scaffold prepared in step 1) to a particle size of 1˜10 μm, then immersing it in a growth factor solution for 12˜24 h, centrifuging, discarding a supernatant, pouring it into a mold for freeze-drying, packaging, and sterilizing by irradiation to obtain bone collagen scaffold material.

8. The preparation method for the bone collagen scaffold material loaded with multiple growth factors according to claim 7, wherein in step 1), when preparing the bone collagen scaffold, defatting solution is a mixture of dichloromethane or chloroform and methanol; the defatting parameters are: temperature: 5˜45° C.: rotation speed: 300˜600 rpm; the defatting solution is a mixture of dichloromethane or chloroform and methanol, with a material-to-liquid ratio of 1:3˜5.

9. The preparation method for the bone collagen scaffold material loaded with multiple growth factors according to claim 7, wherein in step 1), when preparing the bone collagen scaffold, the acidic solution is hydrochloric acid and/or phosphoric acid with a concentration of 0.5˜1 mol/L; the purified water soaking temperature is 0° C., and the purified water is changed every 12 h during soaking; pH is detected when changing the water, and it is qualified when the pH value is 6˜7.5.

10. The preparation method for the bone collagen scaffold material loaded with multiple growth factors according to claim 7, wherein in step 2), for loading growth factors, the concentration of the growth factor solution is:
FGF: 30˜50 ng/ml,
VEGF: 25˜40 ng/ml,
TGF-β: 40˜65 ng/ml,
BMP: 40˜65 ng/ml;
the bone collagen scaffold is added to the growth factor solution with a material-to-liquid ratio of 1:5˜10;
the soaking temperature is 0° C.;
the irradiation sterilization dose is 10˜30 kGy.
